(19) United States
(12) Patent Application Publication
(10) Pub. No.: US 2025/0090173 A1
(43) Pub. Date: Mar. 20, 2025

(54) ACTIVE TUMOR EMBOLIZATION DEVICE

(71) Applicant: KOREA INSTITUTE OF MEDICAL MICROROBOTICS, Gwangju (KR)

(72) Inventors: Jayoung KIM, Daejeon (KR); Kim Tien NGUYEN, Gwangju (KR)

(21) Appl. No.: 18/730,852

(22) PCT Filed: Jan. 31, 2023
(86) PCT No.: PCT/KR2023/001398

§ 371 Date: Jul. 22, 2024

(30) Foreign Application Priority Data
Feb. 9, 2022 (KR) 10-2022-0016932

(51) Int. Cl. A61B 17/12

(52) U.S. Cl. CPC A61B 17/12031; A61B 2017/12086

(57) ABSTRACT

The present invention relates to an active tumor embolization device that actively drives an embolic substance with use of an X-ray system and a magnetic system, thereby being capable of minimizing the use of a catheter for injecting the embolic substance and preventing the necrosis of normal tissues caused by the embolic substance.

### TECHNICAL FIELD

The present invention was made with the support of the Ministry of Trade, Industry and Energy, Republic of Korea, under project identification No. 1415180101 (project No. 20017903), which was conducted in the research project named “Development of medical combination device for active precise delivery of embolic beads for transcatheter arterial chemoembolization and simulator for embolization training to cure liver tumor” in the research program titled “Bio-industry technology development” by Korea Institute of Medical Microrobotics, under the management of the Korea Planning & Evaluation Institute of Industrial Technology, from 1 Apr. 2022 to 31 Dec. 2026.

The present invention relates to an active tumor embolization device capable of minimizing the use of a catheter for injecting an embolic substance and preventing the necrosis of normal tissue caused by the embolic substance by actively actuating the embolic substance through an X-ray system and a magnetic system.

### BACKGROUND ART

Embolization may be performed to block the vessels, through which nutrients or oxygen are supplied to a specific lesion, thereby removing the lesion, or to find and block bleeding vessels to control bleeding, by artificially stopping the blood flow through the use of an embolic substance. Transcatheter arterial chemoembolization for the treatment of hepatocellular carcinoma is most frequently performed. Cerebral aneurysm embolization, bronchial artery embolization, uterine artery embolization, and the like are also performed.

Especially, transcatheter arterial chemoembolization (TACE) is a method of selectively attaining tumor necrosis by finding the hepatic artery, through which oxygen and nutrients necessary for the growth of tumor cells in the liver are supplied, administering an anticancer drug thereto, and stopping the blood flow with the use of an embolic substance.

The liver is an organ that receives dual blood flow supply from the hepatic artery and the hepatic portal vein. Normal liver tissue receives 70-80% of blood flow and 50% of necessary oxygen from the portal vein, while hepatocellular carcinoma is mostly a hypervascular tumor and receives more than 90% of blood from the hepatic artery. Therefore, the injection of a therapeutic substance through the hepatic artery results in the administration of a higher concentration of the therapeutic substance to hepatocellular carcinoma compared with normal liver tissue, and the non-selective stopping of hepatic artery blood flow through the injection of an embolic substance results in severe ischemia in only hepatocellular carcinoma, thereby enabling relatively selective tumor therapy.

However, various complications may occur after TACE. The main complications include liver failure, liver abscess, parenchymal infarction, pulmonary oil embolism, and ischemic cholecystitis, which are reported in less than 5% of cases. The occurrence of these complications cannot be completely prevented due to the nature of the procedure. Most serious complications are caused by main portal vein occlusion, decreased liver function, biliary obstruction, history of biliary surgery, excessive use of Lipiodol, hepatic artery occlusion due to repeated TACE, non-selective embolization, and the like. It is therefore important to prevent the occurrence of complications as much as possible, such as precisely checking the presence or absence of causal factors and considering preventive pretreatment before the procedure and selecting an appropriate procedure method according to the clinical situation during the procedure. For high-risk groups, early detection of complications and prompt response through close follow-up after the procedure are important.

During the procedure, the reflux or influx of an embolic substance into organs other than the liver may cause complications. These complications are caused by the inflow of an anticancer drug or an embolic substance into un-intended vessels due to the failure to recognize anatomical variations in vessel branches in advance. The embolization of the cystic artery causes cholecystitis, which mostly recovers spontaneously, but in very rare cases, cholecystitis may progress to gallbladder necrosis or rupture. In severe cases of celiac artery stenosis or splenomegaly, the blood flow in the common hepatic artery may be reversed, and thus an embolic substance may flow into the splenic artery, causing splenic infarction. The inflow of an embolic substance into the gastroduodenal artery may cause acute pancreatitis and duodenal ulcer, and the inflow of an embolic substance into the accessory left gastric artery or superior artery originating from the hepatic artery may cause gastritis or ulcers. The inflow of an anticancer drug or an embolic substance into the anterior spinal artery during embolization through the axial circulation of the intercostal artery may cause spinal cord damage.

Such movement of an embolic substance outside its intended during embolization may cause various side effects. Therefore, there is an urgent need to develop new procedure devices capable of preventing this movement of the embolic substance through the targeting of the embolic substance, thereby reducing side effects.

### DISCLOSURE OF INVENTION

### Technical Problem

The present inventors have made an effort to derive a device capable of localization and actuation of an embolic substance to reduce side effects after embolization, increasing the convenience of the procedure, and performing efficient progress inspection after the procedure.

As a result, the present inventors have developed an active tumor embolization device capable of localization as well as active actuation of an embolic substance by an X-ray system and a magnetic system.

Accordingly, an aspect of the present invention is to provide an active tumor embolization device.

Another aspect of the present invention is to provide a method for localizing and actuating an embolic substance.

### Solution to Problem

The present invention is directed to an active tumor embolization device including: an X-ray system capable of obtaining X-ray images; a bed unit with a space for a subject to be located; and a magnetic system including an electromagnetic module capable of generating a magnetic field.

Hereinafter, the present invention will be described in more detail.

An embodiment of the present invention is directed to an active tumor embolization device including the following:

an X-ray system including a light irradiator for performing the irradiation of light, a photoelectric conversion substrate for converting light to an electric signal, and a scintillator layer in contact with the photoelectric conversion substrate;

a bed unit disposed between the light irradiator and the photoelectric conversion substrate; and

a magnetic system comprising an electromagnetic module and an actuation module, the electromagnetic module comprising a plate on one side of which a plurality of electromagnets are arranged, with an RF coil part disposed apart from the electromagnets, the actuation module being fastened to the other side of the plate through a fastening element.

In the present invention, the X-ray system may include a light irradiator for performing the irradiation of light, a photoelectric conversion substrate for converting light to an electric signal, and a scintillator layer in contact with the photoelectric conversion substrate, but is not limited thereto.

In the present invention, the X-ray system may take an image of the inside of a subject by using computed radiography (CR) or digital radiography (DR), but is not limited thereto.

In the present invention, the subject may be a human or mammal, but is not limited thereto.

In the present invention, the X-ray system may be implemented as a planar X-ray system based on solid imaging elements, such as active matrix, CCD, and CMOS, but is not limited thereto.

In the present invention, the planar X-ray system may include: a photoelectric conversion substrate for converting light to an electric signal; and a scintillator layer in contact with the photoelectric conversion substrate, but is not limited thereto.

In the present invention, in the planar X-ray system, the scintillator layer is irradiated with X-rays to convert the X-rays into light, and the converted light is incident on the photoelectric conversion substrate to convert the light into an electrical signal, and thus an X-ray image or real-time X-ray image may be outputted as a digital signal, but the planar X-ray system is not limited thereto.

In the present invention, the light irradiator and the scintillator layer may be disposed to face each other relative to the bed unit, but are not limited thereto.

In the present invention, the bed unit may be included between the light irradiator and the photoelectric conversion substrate, but is not limited thereto.

In the present invention, the light from the light irradiator may penetrate the bed unit, but is not limited thereto.

In the present invention, the bed unit may have a predetermined area where the subject can be positioned, and for example, the bed unit may have a flat area so that a human body can lie down thereon, but is not limited thereto.

In the present invention, the magnetic system may include an electromagnetic module and an actuation module, the electromagnetic module including a plate on one side of which a plurality of electromagnets are arranged, with an RF coil part disposed apart from the electromagnets, the actuation module being fastened to the other side of the plate through a fastening element, but is not limited thereto.

In the present invention, the electromagnetic module may include a plate on one side of which a plurality of electromagnets are arranged, with an RF coil part disposed apart from the electromagnets, but is not limited thereto.

In the present invention, the RF coil part may include a receiving coil (Rx coil) and a transmitting coil (Tx coil) disposed along the outer circumference of the Rx coil, but is not limited thereto.

In the present invention, the Rx coil may be a coil that is exclusively used for receiving wireless signals, but is not limited thereto.

In the present invention, the Tx coil may be a coil that is exclusively used for transmitting wireless signals, but is not limited thereto.

In the present invention, the outer circumference of the Rx coil may be adjacent to the inner circumference of the Tx coil, but is not limited thereto.

In the present invention, the RF coil part may be disposed to face the actuation module relative to the electromagnets, but is not limited thereto.

In the present disclosure, the electromagnets may correspond to at least one selected from the group consisting of a solenoid coil, a circular coil, a square coil, and a saddle coil, but are not limited thereto.

In the present invention, the electromagnets may generate a magnetic field by receiving current, and thus can generate and control field free point (FFP) or field free line (FFL), but is not limited thereto.

As used herein, the term “field free point” or “FFP” may mean a point where the magnetic field strength is zero in the magnetic field generated by an electromagnet.

As used herein, the term “field free line” or “FFL” may mean a line where the magnetic field strength is zero in the magnetic field generated by an electromagnet.

In the present invention, the electromagnets generate field free points or field free lines to scan the subject, and magnetic nanoparticles contained in the embolic substance may generate a reflection signal when receiving a magnetic field. Considering that the closer the field free point or field free line is to the magnetic nanoparticle, the stronger the reflection signal, the analysis of the reflection signal can be used to recognize the current position of the magnetic nanoparticle.

In the present invention, the number of electromagnets may be 2 or more, 4 or more, 6 or more, 8 or more, or 10 or more, but is not limited thereto.

In an embodiment of the present invention, six electromagnets may be present, and each electromagnet may be independently supplied with current from a separately provided power supply, but is not limited thereto.

In the present invention, the plurality of electromagnets may be arranged on one side of the plate, but are not limited thereto.

In the present invention, the shape of the plate may be linear or curved, but is not limited thereto.

In an embodiment of the present invention, the plate may be curved to have a predetermined curvature such that the long axes of the plurality of arranged electromagnets converge at a single point in space, but is not limited thereto.

In the present invention, the actuation module may be fastened to the other side of the plate through a fastening element, but is not limited thereto.

In the present invention, a plurality of electromagnets may be arranged on one side of the plate, and the other side of the plate may be fastened to the actuation module through the fastening element, but the plate is not limited thereto.

In an embodiment of the present invention, the other side of the plate may be further provided with a plate actuation member.

In the present invention, the plate actuation member may be connected to the fastening element, on the other side of the plate, such that the plate is slidably actuated.

In the present invention, the plate actuation member may be disposed across the other side of the plate, but is not limited to thereto.

In the present invention, the actuation module may include a fastening arm capable of rotating actuation relative to the long axis thereof, but is not limited thereto.

In the present invention, one side of the fastening arm may be coupled to the electromagnetic module through the fastening element, and the other side thereof may be coupled to the actuation module, but is not limited thereto.

In an embodiment of the present invention, the actuation module may further include a fastening arm actuation member to enable the fastening arm to be slidably actuated in the long axis direction thereof, but is not limited thereto.

In the present invention, the fastening arm actuation member may have a hollow interior for the fastening arm to enter, and thus the fastening arm may enter the hollow interior, but is not limited thereto. The length of the fastening arm may be adjusted through such a structure.

In an embodiment of the present invention, the long axis direction of the fastening arm may be parallel with the ground.

In an embodiment of the present invention, the fastening arm actuation member may be coupled to a vertical support such that the fastening arm actuation member is slidably actuated in a direction vertical to the ground, but is not limited thereto.

In the present invention, one point in space, toward which the long axes of the electromagnets are directed, may be changed through sliding actuation of each of the fastening element, the fastening arm, the fastening arm actuation member, and the vertical support.

In the present invention, the actuation module may further include moving members at the bottom.

In the present invention, the moving members may be moving wheels equipped with anchors, but are not limited thereto.

In the present invention, the actuation module may further include a controller, but is not limited thereto.

In the present invention, the controller receives the manipulation information from a user to transmit the information to the fastening element, fastening arm, fastening arm actuation member, and vertical support, which are included in the actuation module, thereby controlling the actuation thereof, but is not limited thereto.

In the present invention, the device may further include a display unit, but is not limited thereto.

In the present invention, the display unit may be attached to the device of the present invention or may be separately provided, but is not limited thereto.

In the present invention, the display unit may be connected to communicate with at least one of the X-ray system and the magnetic system, but is not limited thereto. The type of communication may be wired or wireless, but is not limited thereto.

In an embodiment of the present invention, the actuation module may further include a catheter for delivering the embolic substance to the subject, but is not limited thereto.

In the present invention, the device may be used for an embolization procedure.

Another embodiment of the present invention is directed to a method for providing information necessary to determine the extent of embolization, the method comprising:

an X-ray irradiation step of irradiating a subject with X-rays so that the X-rays penetrate the subject to reach a scintillator layer, thereby creating an X-ray image;

a first scan step of searching an embolic substance by using field free point or field free line;

a reflection signal reception step of receiving a reflection signal reflected from the embolic substance through an RF coil part;

a magnetic field application step of applying a magnetic field to the embolic substance so that magnetic force acts in a direction crossing the movement direction of the embolic substance; and

a second scan step of searching the embolic substance in a target area by using field free point or field free line and then creating an embolic substance image.

In the present invention, the X-ray irradiation step may be irradiating a subject with X-rays so that the X-rays penetrate the subject to reach a scintillator layer, thereby creating X-ray images, but is not limited thereto.

In the present invention, the first scan step may be performed by an electromagnetic module including a plate on one side of which a plurality of electromagnets are arranged to face towards the subject, but is not limited thereto.

In the present invention, the first scan step may be searching the embolic substance by generating field free point or field free line.

In the present invention, the first scan step may be searching the position of the embolic substance before the magnetic field application step.

In the present invention, the embolic substance may contain magnetic nanoparticles, but is not limited thereto.

In the present invention, the magnetic nanoparticles may have magnetic properties that allow their translocation using a magnetic field induced by an external apparatus, but are not limited thereto.

In the present invention, the magnetic nanoparticles may further load a drug, but are not limited thereto.

In the present invention, the drug may be at least one selected from the group consisting of doxorubicine, epirubicin, gemsitabin, cisplatin, carboplatin, procarbazine, cyclophosphamide, dactinomycin, daunorubicin, etoposide, tamoxifen, mitomycin, bleomycin, plicomycin, transplatinum, vinblastine, and methotrexate. For example, the drug may be doxorubicin, but is not limited thereto.

In the present invention, the embolic substance may further contain at least one contrast agent selected from the group consisting of iodinated contrast agents and barium contrast agents, but is not limited thereto.

In the present invention, the reflection signal reception step may be receiving a reflection signal reflected from the embolic substance through an RF coil part. For example, the Rx coil may receive a reflection signal generated by magnetic nanoparticles, contained in the embolic substance, through the application of a magnetic field, but is not limited thereto.

In the present invention, the reflection signal reception step may be recognizing the current position the magnetic nanoparticle by analyzing the intensity of the reflection signal considering that the closer the field free point or field free line is to the magnetic nanoparticle, the stronger the intensity of the reflection signal, but is not limited thereto.

In the present invention, the reflection signal reception step may further include a position display step of displaying the position of the embolic substance on the X-ray image after the recognizing of the position of the embolic substance, but is not limited thereto.

In the present invention, there may be no relationship in temporal order between the X-ray irradiation step and the first scan step.

In an embodiment of the present invention, the first reflection signal reception step may be receiving the reflection signal generated by the magnetic nanoparticles contained in the embolic substance, through the application of a magnetic field, before the magnetic field application step, but is not limited thereto.

In the present invention, the magnetic field application step may be applying a magnetic field to the embolic substance so that magnetic force acts in a direction crossing the movement direction of the embolic substance, in consideration of the movement direction of the embolic substance and branched vessels before the application of the magnetic field to the embolic substance, but is not limited thereto.

Therefore, the embolic substance may not move in the branching directions of the vessel, and the embolic substance can move following the blood flow.

In an embodiment of the present invention, the magnetic field application step may be performed by a magnetic system including an electromagnetic module and an actuation module, the electromagnetic module including a plate on one end of which a plurality of electromagnets are arranged, with an RF coil part disposed apart from the electromagnets, the actuation module being fastened to the other side of the plate through a fastening element, but is not limited thereto.

In an embodiment of the present invention, the magnetic field application step may be moving the embolic substance by using X-ray images, obtained from the X-ray irradiation step, to determine a magnetic field in a direction cross the movement direction of the embolic substance, but is not limited thereto.

In the present invention, the second scan step may be searching the embolic substance in a target area by using magnetic field free point or magnetic field free line, and then creating an embolic substance image.

In the present invention, the embolic substance image may contain at least one selected from the group consisting of information on targeting efficiency of the embolic substance, distribution of embolic particles, and decomposition of embolic particles. For example, the embolic substance image may contain all of information on targeting efficiency of the embolic substance, information on distribution of embolic particles, and information on decomposition of embolic particles, but is not limited thereto.

In the present invention, the embolic substance image may be utilized in the doctor's clinical judgment, leading to the determination of the extent of embolization, but is not limited thereto.

In an embodiment of the present invention, the second scan step may further include a second reflection signal reception step of receiving a reflection signal reflected from the embolic substance after the magnetic field application step, thereby creating the embolic substance image.

### Advantageous Effects of Invention

The present invention is directed to an active tumor embolization device capable of minimizing the use of a catheter for injection of an embolic substance and preventing the necrosis of normal tissue due to the embolic substance by actively actuating the embolic substance through an X-ray system and a magnetic system. The present invention can be used to facilitate the movement of the embolic substance to a target site even without the catheter accessing deep into the vessel, thereby enabling targeting of the embolic substance to a tumor site and thus preventing damage to normal tissue.

Furthermore, the path of the embolic substance can be checked in real time even without the catheter, for injection of the embolic substance, assessing deep into the vessel, so even an unskilled operator can perform an embolization procedure at the same level as an expert.

Furthermore, a non-proximity procedure can be achieved, thereby increasing the operator's accessibility to a patient and preventing the operator's exposure to radiation, and the extent of embolization can be easily inspected by using the magnetic system, thereby reducing additional medical processes, such as CT or MRI, leading to a reduction in cost and time and an improvement in patient convenience.

### BRIEF DESCRIPTION OF DRAWINGS

FIG. 1 is a schematic diagram illustrating the localization and actuation of an embolic substance injected into a subject by using an active tumor embolization device according to an embodiment of the present invention.

FIG. 2 is a schematic diagram illustrating a detailed configuration of a magnetic system according to an embodiment of the present invention.

FIG. 3 is a schematic diagram illustrating the detailed configurations of an electromagnet module and an actuation module and the operation of the actuation module to move the electromagnet module according to an embodiment of the present invention.

FIG. 4 is a flow chart illustrating the localization of an embolic substance and the driving process through the use of the device according to an embodiment of the present invention.

FIG. 5 is a flow chart illustrating magnetic localization using the device according to an embodiment of the present invention.

FIG. 6 is a flow chart illustrating X-ray and EMM coordinate matching using the device according to an embodiment of the present invention.

FIG. 7 is a flow chart illustrating the setting of a path of an embolic substance using the device according to an embodiment of the present invention.

FIG. 8 is a flow chart illustrating the actuation of magnetic nanoparticles contained in an embolic substance by using the device according to an embodiment of the present invention.

FIG. 9 is a flow chart illustrating an inspection process of the embolization degree by using the device according to an embodiment of the present invention.

### BEST MODE FOR CARRYING OUT THE INVENTION

An active tumor embolization device, including:

an X-ray system including a light irradiator for performing the irradiation of light, a photoelectric conversion substrate for converting light to an electric signal, and a scintillator layer in contact with the photoelectric conversion substrate;

a bed unit disposed between the light irradiator and the photoelectric conversion substrate; and

a magnetic system including an electromagnetic module and an actuation module, the electromagnetic module including a plate on one side of which a plurality of electromagnets are arranged, with an RF coil part disposed apart from the electromagnets, the actuation module being fastened to the other side of the plate through a fastening element.

### BEST MODE FOR CARRYING OUT THE INVENTION

Hereinafter, the present invention will be described in more detail with reference to exemplary embodiments. These exemplary embodiments are provided only for the purpose of illustrating the present invention in more detail, and therefore, according to the purpose of the present invention, it would be apparent to a person skilled in the art that these exemplary embodiments are not construed to limit the scope of the present invention.

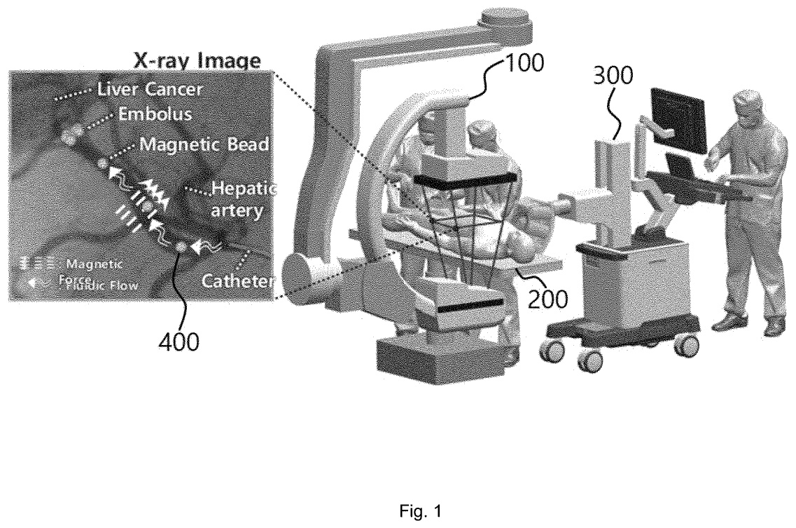

FIG. 1 is a schematic diagram illustrating the localization and actuation of an embolic substance injected into a subject by using an active tumor embolization device according to an embodiment of the present invention.

Referring to FIG. 1, the active tumor embolization device according to an embodiment of the present invention may include an X-ray system 100, a bed unit 200, and a magnetic system 300.

An embolic substance containing magnetic nanoparticles 400 may be injected into the vessel of a patient through a catheter by an operator. The appearance of the magnetic nanoparticles 400 and vessels can be confirmed through an X-ray image.

The magnetic nanoparticles 400 can basically move along the blood flow. However, the magnetic nanoparticles do not necessarily reach the liver cancer through the feeding artery. The device of the present invention can be used to apply a magnetic field to the magnetic nanoparticles 400 so that the magnetic force acts in the opposite direction of the branched vessels branching from the feeding artery to prevent the magnetic nanoparticles 400 from moving into the branched vessels. Therefore, the magnetic nanoparticles 400 can move towards a target liver cancer.

In the X-ray system 100 of the present invention, a light irradiator and a photoelectric conversion substrate are disposed above and below the bed unit 200, respectively, thereby facilitating the operator's accessibility to the patient.

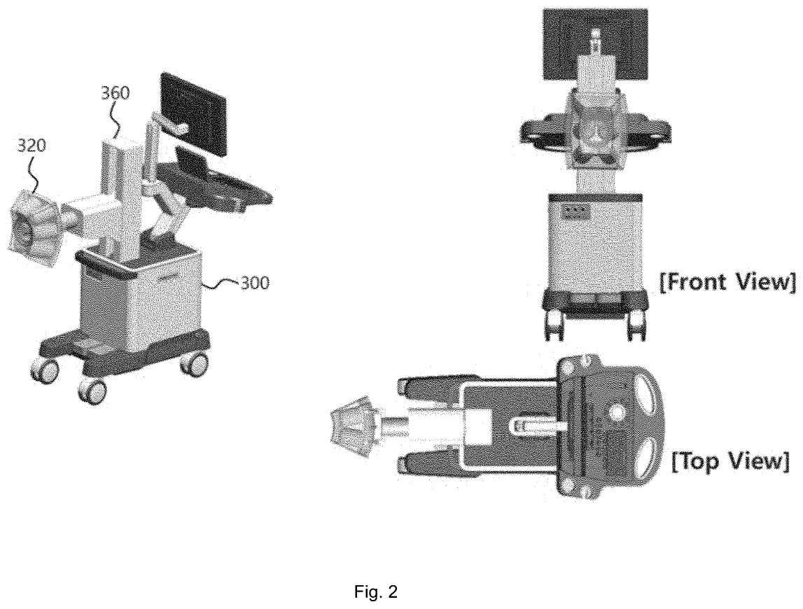

FIG. 2 is a schematic diagram illustrating a detailed configuration of the magnetic system according to an embodiment of the present invention.

Referring to FIG. 2, the magnetic system 300 of the present invention may include an electromagnet module 320 and an actuation module 360.

The actuation module 360 may be coupled to a cabinet and may further include a display such as a monitor, and a controller including a keyboard, mouse, and/or joystick. An operator may check the current position of the magnetic nanoparticles 400 by using an X-ray image obtained by the X-ray system 100 and a reflection signal of the magnetic nanoparticles 400 obtained by the magnetic system 300. The operator may control the actuation module 360 in real time by manipulating the controller.

Additionally, the cabinet may further include moving members such as moving wheels, at the bottom, thereby improving the movement convenience of the magnetic system 300.

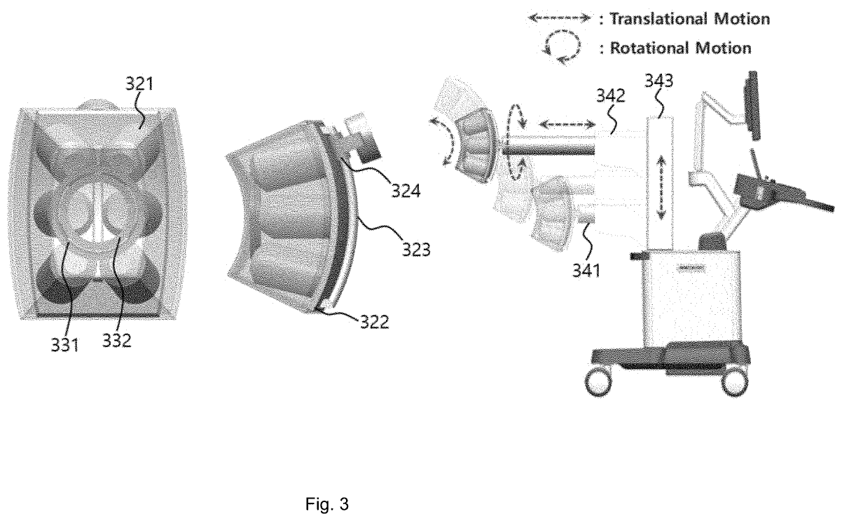

FIG. 3 is a schematic diagram illustrating the detailed configurations of the electromagnet module 320 and the actuation module 360 and the operation of the actuation module to move the electromagnet module 320 according to an embodiment of the present invention.

Referring to FIG. 3, the electromagnet module 320 according to an embodiment of the present invention may include a plurality of electromagnets 321, a plate 322 on which the plurality of electromagnets are arranged, a Tx coil 331, and an Rx coil 332.

The Rx coil may be used exclusively for receiving wireless signals, and the Rx coil may be used exclusively for transmitting wireless signals. The outer circumference of the Rx coil may be adjacent to the inner circumference of the Tx coil. The Tx coil and Rx coil 331 and 332 according to an embodiment of the present invention may be disposed in the direction of the patient to face the actuation module 360, relative to the electromagnets 321.

The number of the electromagnets 321 according to an embodiment of the present invention may be six.

The six electromagnets 321 according to an embodiment of the present invention may be arranged on one side of the plate 322, which is curved to have a predetermined curvature such that the long axes of the electromagnets converge at a single point in space. Particularly, the six electromagnets may be independently supplied with power from separately connected power supplies to generate a magnetic field.

A plate actuation member 323 may be further provided on the other side of the plate 322. The plate actuation member 323 may be disposed across the plate 322 to be connected to a fastening element 324 such that the plate 322 is slidably actuated. The plate actuation member 323 according to an embodiment of the present invention may be disposed to cross in the longitudinal sectional direction of the plate 322. Therefore, the plate 322 curved to have a predetermined curvature is slidably actuated.

The fastening element 324 may be connected to the plate actuation member 323 through a separate actuation member such that the plate 322 is slidably actuated, but is not limited thereto.

A material for the plate 322 and plate actuation member 323 is not particularly limited, and any material that can possess a predetermined rigidity to withstand the weight and load of the electromagnetic module 320 and actuation module 360 of the present invention may be used without limitation.

The actuation module 360 according to an embodiment of the present invention may further include: fastening arm 341 connected to the fastening element; a fastening arm actuation member 342 have a hollow interior where the fastening arm can enter; and a vertical support 343 to which the fastening arm driving member 342 is connected to enable sliding actuation in a direction vertical to the ground.

The fastening arm 341 may be slidably actuated by insertion into or discharge from the fastening arm actuation member 342 having the hollow inside. Additionally, the fastening arm 341 may be rotatably actuated relative to its long axis.

The fastening arm actuation member 342 may be connected to the vertical support 343 such that the fastening arm actuation member is slidably actuated in a direction vertical to the ground.

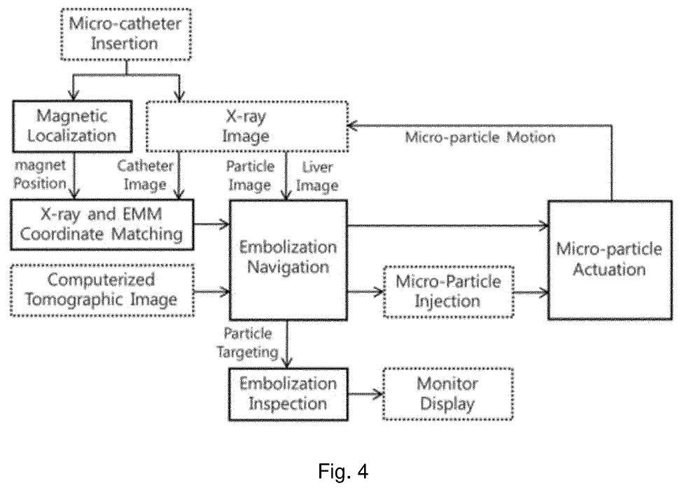

FIG. 4 is a flow chart illustrating an active embolization process using the device according to an embodiment of the present invention.

Referring to FIG. 4, as for a method for localizing and actuating an embolic substance according to the present invention, micro-catheter insertion was conducted, and then X-ray image creation and magnetic localization are conducted (Magnetic Localization). The shape and insertion location of the micro-catheter labeled with a magnetic marker may be identified from the obtained X-ray image, and the coordinates of the magnetic system and the X-ray system are matched using the X-ray image (X-ray and EMM Coordinate Matching).

In this procedure, computerized tomographic images captured by CT imaging device were conventionally used, but no CT images may be needed in the coordinate matching process of the present invention.

Next, the path of the embolic substance is set, and a magnetic field is generated to enable the embolic substance to move to the cancer. In the use of the active tumor embolization device according to the present invention, unlike the prior art, the embolic substance may be injected in advance. When the embolic substance moves, an X-ray image is re-generated, and the position of the embolic substance is checked by rescanning in order to inspect whether the embolic substance has moved correctly towards the cancer, and then an embolic substance image is created.

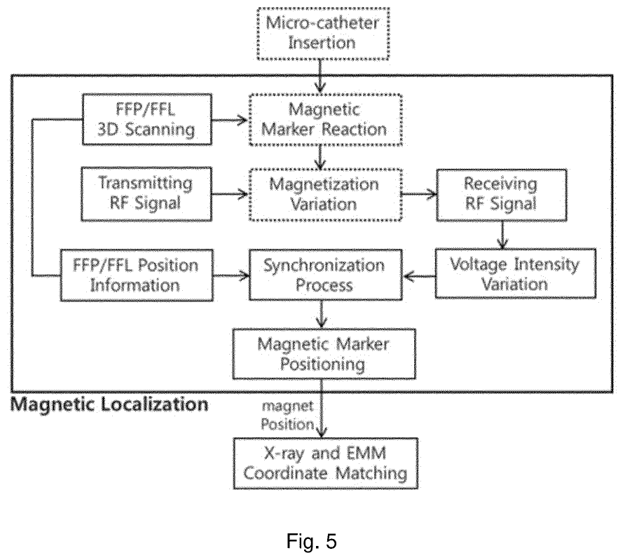

FIG. 5 is a flow chart illustrating magnetic localization using the device according to an embodiment of the present invention.

In FIG. 5, a tube of the micro-catheter is equipped with a magnetic marker, and the magnetic marker may be utilized for magnetic localization and X-ray-based localization to match the coordinates on the magnetic system and X-ray images. Upon scanning using field free point or field free line (FFP/FFL) in the three-dimensional space through the electromagnets of the magnetic system, the degree of magnetization of the magnetic marker varies depending on the generation location of FFP/FFL (Magnetic Marker Reaction and Magnetization Variation). Particularly, the localization is conducted using the intensities of RF signals transmitted (Tx coil) and received (Rx coil).

Specifically, when a field map of FFP/FFL is generated in space, the RF signal intensity (voltage intensity) is weakest if the magnetic marker is positioned in the area where the magnetic field is strongest, and the RF signal intensity is strongest if the magnetic marker is positioned in the area where the magnetic field is 0. Particularly, the area where the magnetic field is 0 corresponds to FFP/FFL, and thus, the location of FFP/FFL generation is controlled to induce the area where the RF signal intensity is strongest, which is converted into the position of the magnetic marker (FFP/FFL 3D Scanning).

Therefore, a synchronization process of FFP/FFL position information and RF signal intensity information (voltage intensity variation) is conducted to recognize magnetic marker positioning.

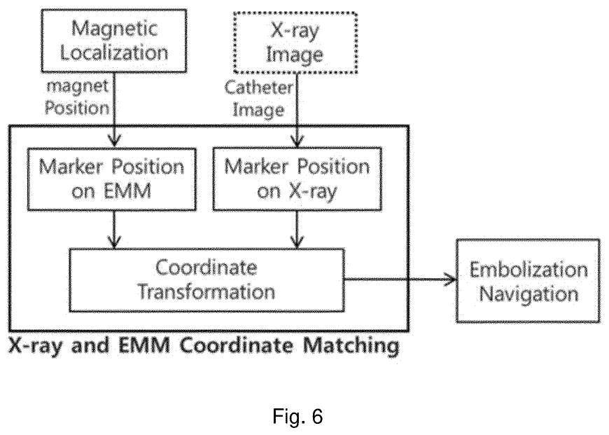

FIG. 6 is a flow chart illustrating X-ray and EMM coordinate matching using the device according to an embodiment of the present invention.

Referring to FIG. 6, the current position of the micro-catheter may be recognized through magnetic localization, and coordinates matching thereto are displayed on the X-ray image, thereby proceeding with setting the embolic substance path.

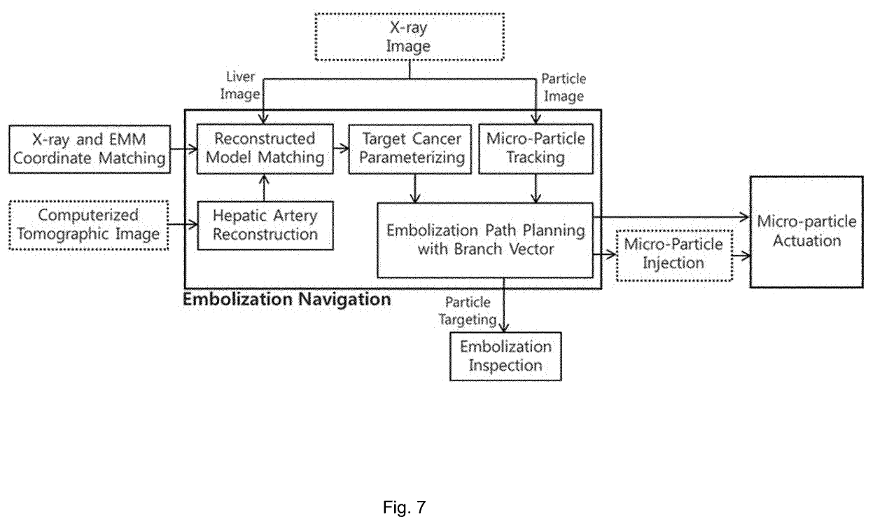

FIG. 7 is a flow chart illustrating the setting of the embolic substance path using the device according to an embodiment of the present invention.

Referring to FIG. 7, the setting of the embolic substance path begins by matching a 3D vessel model created from a computerized tomographic image (CT image) with an X-ray image. Specifically, the matching may be conducted by feature matching of the 3D vessel model and the X-ray image (Reconstructed Model Matching).

Next, the position, size, and volume of the target cancer are converted into figures based on the X-ray coordinates (Target Cancer Parameterizing).

Last, the path of embolic particles is planned relative to the coordinates on the X-ray image. Particularly, the vessel on the path is not a single vessel but has various vessel branches, and thus there needs a path plan based on the location information of the vessel branches (branch vector) for precise super-selection (Embolization Path Planning with Branch Vector).

In conclusion, the path of the embolic substance may be created considering the position information of vessel branches.

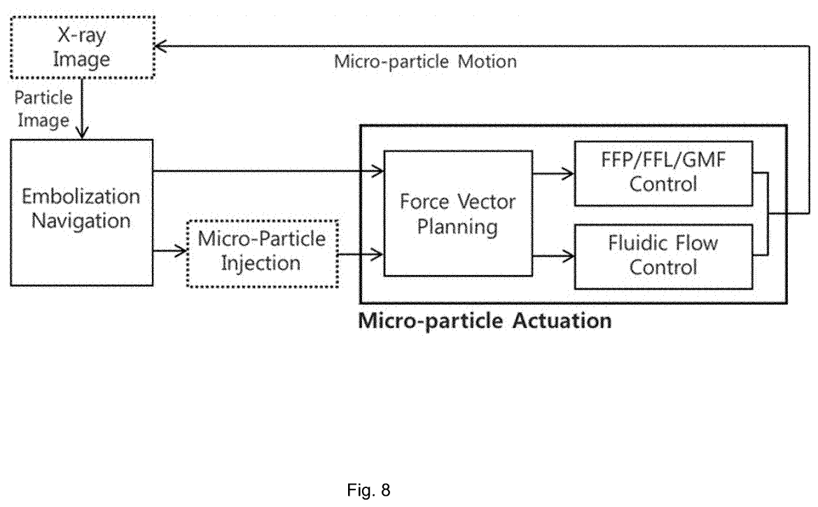

FIG. 8 is a flow chart illustrating the actuation of magnetic nanoparticles contained in the embolic substance by using the device according to an embodiment of the present invention.

Referring to FIG. 8, the device according to an embodiment of the present invention sets the path of the embolic substance by using the obtained X-ray image. To allow the embolic substance to continuously move towards the feeding artery, the magnetic system of the present invention can apply a magnetic field to cross the progression direction of the embolic substance to prevent the embolic substance from moving into arterioles or capillaries. In this procedure, consideration needs to be made on both the blood flow and direction of the vessel where the embolic substance is located. The reason is that the movement of the embolic substance is implemented by the fluidic flow and magnetic force (FFP/FFL/GMF).

Force vector planning is an actuation force planning process for movement and self-steering (direction change) of the embolic substance according to the planned path. The planned 3D actuation force information is utilized for FFP/FFL/GMF Control and Fluidic Flow Control to control the embolic substance. FFP/FFL/GMF Control may mean controlling the direction and magnitude of the three-dimensional magnetic force to steer the embolic substance loading magnetic particles in a direction of the target path through FFP/FFL/GMF control. Fluidic Flow Control is a fluid flow control process for moving the embolic substance, whereby the movement of the embolic substance may be controlled by adjusting the rate and amount of a fluid (saline, etc.) through a separate catheter channel.

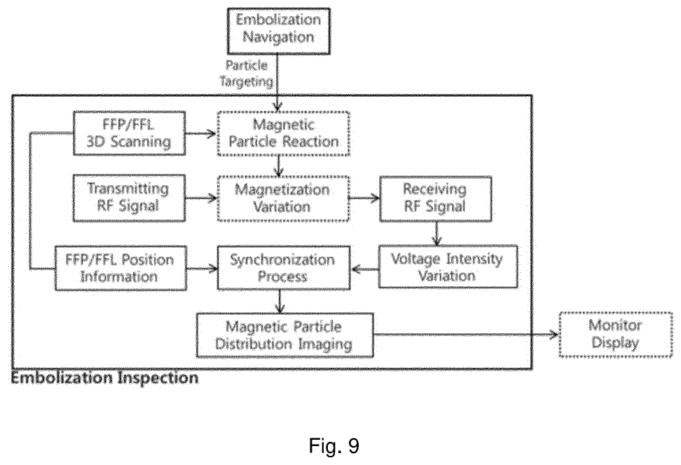

FIG. 9 is a flow chart illustrating an inspection process of the extent of embolization by using the device according to an embodiment of the present invention.

Referring to FIG. 9, as for the inspection process of the extent of embolization, the embolic substance path is set and the field free point or field free line is generated, thereby searching the current position of the magnetic nanoparticles contained in the embolic substance. The extent of embolization (embolization retention and particle distribution) is inspected by receiving a reflection signal generated from magnetic particles through the use of an RF coil part included in the electromagnetic module of the present invention and checking the current position of the magnetic nanoparticles through the reflection signal (Embolization Inspection).

Particularly, the device according to the present invention can be used to create an embolic substance image through magnetic particle distribution imaging, thereby providing numerical information and visualization images on targeting efficiency of the embolic substance, distribution of the embolic particles, and decomposition of the embolic particles. Therefore, unlike the conventional art that clinical specialists relied solely on their experience and subjective judgment to assess the extent of embolization, the present invention allows the use of visualized objective information, thus ensuring the efficiency and consistency of clinical verification.

### Industrial Applicability

The present invention is directed to an active tumor embolization device capable of minimizing the use of a catheter for injection of an embolic substance and preventing the necrosis of normal tissue due to the embolic substance by actively actuating the embolic substance through an X-ray system and a magnetic system.

What is claimed is:

1. An active tumor embolization device, comprising:
an X-ray system comprising a light irradiator for performing the irradiation of light, a photoelectric conversion substrate for converting light to an electric signal, and a scintillator layer in contact with the photoelectric conversion substrate;
a bed unit disposed between the light irradiator and the photoelectric conversion substrate; and
a magnetic system comprising an electromagnetic module and an actuation module, the electromagnetic module comprising a plate on one side of which a plurality of electromagnets are arranged, with an RF coil part disposed apart from the electromagnets, the actuation module being fastened to the other side of the plate through a fastening element.

2. The active tumor embolization device of claim 1, wherein the electromagnets correspond to at least one selected from the group consisting of a solenoid coil, a circular coil, a square coil, and a saddle coil.

3. The active tumor embolization device of claim 1, wherein the RF coil part comprises an Rx coil and a Tx coil disposed along the outer circumference of the Rx coil, the RF coil part being disposed to face the actuation module relative to the electromagnets.

4. The active tumor embolization device of claim 1, wherein the plate is curved to have a predetermined curvature such that the long axes of the plurality of arranged electromagnets converge at a single point in space.

5. The active tumor embolization device of claim 1, wherein the other side of the plate is further provided with a plate actuation member connected to the fastening element such that the plate is slidably actuated.

6. The active tumor embolization device of claim 1, wherein the actuation module comprises a fastening arm enabling rotating actuation around the long axis thereof, and further comprises a fastening arm actuation member enabling the fastening arm to be slidably actuated in a long axis direction thereof.

7. The active tumor embolization device of claim 6, wherein the long axis direction of the fastening arm is parallel with the ground, and the fastening arm actuation member is coupled to a vertical support to enable sliding actuation in a direction vertical to the ground.

8. The active tumor embolization device of claim 1, wherein the actuation module further comprises moving members.

9. The active tumor embolization device of claim 1, wherein the actuation module further comprises a display unit connected to communicate with at least one of the X-ray system and the magnetic system.

10. A method for providing information necessary to determine the extent of embolization, the method comprising:
an X-ray irradiation step of irradiating a subject with X-rays so that the X-rays penetrate the subject to reach a scintillator layer, thereby creating an X-ray image;
a first scan step of searching an embolic substance by using field free point or field free line;
a reflection signal reception step of receiving a reflection signal reflected from the embolic substance through an RF coil part;
a magnetic field application step of applying a magnetic field to the embolic substance so that magnetic force acts in a direction crossing the movement direction of the embolic substance; and
a second scan step of searching the embolic substance in a target area by using field free point or field free line and then creating an embolic substance image.

11. The method of claim 10, wherein the scan steps are performed by an electromagnetic module comprising a plate on one side of which a plurality of electromagnets are arranged towards the subject.

12. The method of claim 10, wherein the embolic substance contains magnetic nanoparticles.

13. The method of claim 12, wherein the magnetic nanoparticles further load a drug, the drug being at least one selected from the group consisting of doxorubicine, epirubicin, gemsitabin, cisplatin, carboplatin, procarbazine, cyclophosphamide, dactinomycin, daunorubicin, etoposide, tamoxifen, mitomycin, bleomycin, plicomycin, transplatinum, vinblastine, and methotrexate.

14. The method of claim 10, wherein the magnetic field application step is performed by a magnetic system comprising an electromagnetic module and an actuation module, the electromagnetic module comprising a plate on one side of which a plurality of electromagnets are arranged, with an RF coil part disposed apart from the electromagnets, the actuation module being fastened to the other side of the plate through a fastening element.

15. The method of claim 10, wherein in the magnetic field application step, the embolic substance is moved by using an X-ray image obtained from the X-ray irradiation step to determine a magnetic field in a direction cross the movement direction of the embolic substance.

16. The method of claim 10, wherein the embolic substance image contains at least one selected from the group consisting of information on targeting efficiency of the embolic substance, distribution of embolic particles, and decomposition degree of embolic particles.
